(19) United States
(12) Patent Application Publication
(10) Pub. No.: US 2009/0093547 A1
(43) Pub. Date: Apr. 9, 2009

(54) Compositions and Methods for Treating Premature Ejaculation

(75) Inventors: James M. Corbitt, Tucker, GA (US); Brian Adams, Atlanta, GA (US); Larry Dillaha, Atlanta, GA (US)

Correspondence Address: Pabst Patent Group LLP, 1545 PEACHTREE STREET NE, SUITE 320, ATLANTA, GA, 30309, US

(73) Assignee: Sciele Pharma, Inc.

(21) Appl. No.: 11/869,280

(22) Filed: Oct. 9, 2007

(51) Int. Cl. A61K 31/16; A61P 15/12

(52) U.S. Cl. 514/626

(57) ABSTRACT

Compositions and methods for the treatment of premature ejaculation have been developed. It has been discovered that premature ejaculation can be treated by administering a local anesthetic to the glans penis. The local anesthetic may be formulated as an emulsion, lotion, paste, gel, cream, ointment, shea butter, suspension, solution, balm, salve, foam, or in the form of a pump spray to achieve local anesthesia of the dorsal neurons of the penis.

### FIELD OF THE INVENTION

Compositions and methods for treating premature ejaculation have been developed, which are useful for desensitizing the dorsal nerves of the penis.

### BACKGROUND OF THE INVENTION

Premature ejaculation (PE) remains the most common sexual dysfunction in men. Although progress is being made in elucidating its causal mechanisms, the disparate variety of techniques and drugs available for its treatment has met only limited success. For instance, behavioral interventions were reported to be effective with initially satisfactory results in the vicinity of 90%; unfortunately, the success rates decline markedly to less than 25% after 3 years. Systemic drugs have proven to have varying levels of efficacy, but are plagued by side effects that are generally considered unacceptable, in the long term, in otherwise healthy men. Equally discouraging, the effect of these medications is temporary since the symptoms return upon their discontinuation.

The early Freudian-based causal hypothesis of PE has been revised and largely abandoned in favor of physiological facts. A heightened sensitivity of the glans to tactile stimuli is currently believed to be a fundamental initiating factor in PE. Therefore, it is possible that reducing the sensitivity of the glans could translate into a delaying effect on intravaginal ejaculation latency time (IVELT) without adversely affecting the sensation of ejaculation. A drastic approach to desensitization involves “neurotomy” of the dorsal nerves of the penis. This invasive and irreversible measure is reported to be effective but has failed to gain wide support in the medical community.

Topical formulations for the treatment of PE have also been evaluated. Stud 100® (available from Pound International, Ltd, England), an over the counter formulations containing 9.6% w/v lidocaine HCl, is marketed as a treatment for PE. This product contains an ionized form of lidocaine in order to increase the solubility of the lidocaine in the delivery vehicle at physiological pH. However, lidocaine salts do not effectively penetrate keratinized and non-keratinized skin. Thus, this product is considered to be ineffective for the treatment of PE. In fact, no controlled clinical trials have been conducted showing that this product is effective for the treatment of PE.

Xylocalne®, a lidocaine spray, is used primarily for the treatment of sore throats. Xylocalne contains ethanol as a solvent and dichloroethane and trichloroethane as propellants. Xylocalne is not ideal for use in topical applications because the propellants can cause irritation of the mucosal membranes, such as the glans penis.

EMLA, a cream containing a local anesthetic, may be an alternative treatment for PE. However, the active ingredients of EMLA take a long time to penetrate the glans so that the formulations would need to be applied well in advance of sexual activity, which can limit spontaneity. Further, the cream is greasy, difficult to administer, and requires the use of a condom in order to prevent partner transfer. Finally, as EMLA penetrates keratinized skin, it numbs the whole of the penis, not just the glans, which can compromise sexual satisfaction.

There exists a need for improved alternatives for the effective treatment of PE.

Therefore, it is an object of the invention to provide compositions for the effective treatment of PE and methods of use thereof.

It is another object of the invention to provide local compositions containing an effective amount of one or more local anesthetics to cause desensitization of the neurons of the glans penis and methods of making and using thereof.

### SUMMARY OF THE INVENTION

Compositions for the treatment of premature ejaculation (i.e. to delay or have control over ejaculation) and methods of making and using thereof have been developed. The compositions contain an effective amount of one or more anesthetics for local administration to the glans penis. The compositions may be formulated as an emulsion, lotion, paste, gel, cream, ointment, shea butter, suspension, solution, balm, salve, foam, or pump spray to achieve local anesthesia of the dorsal neurons of the penis.

The compositions may further contain one or more additional pharmaceutically active agents. Suitable classes of active agents include, but are not limited to, antimicrobial agents (antibacterial and antifungal agents, and/or antiprotozoal agents), anti-inflammatory agents (non-steroidal or steroidal agents), vasodilator agents, anti-oxidants, vitamins, and hormones.

### DETAILED DESCRIPTION OF THE INVENTION

### I. Definitions

“Water soluble” as used herein refers to substances that have a solubility of greater than or equal to 5 g/100 ml water.

“Lipid soluble” as used herein refers to substances that have a solubility of greater than or equal to 5 g/100 ml in a hydrophobic liquid such as castor oil.

“Hydrophilic” as used herein refers to substances that have strongly polar groups that readily interact with water.

“Lipophilic” as used herein refers to compounds having an affinity for lipids.

“Amphiphilic” as used herein refers to a molecule combining hydrophilic and lipophilic (hydrophobic) properties

“Hydrophobic” as used herein refers to substances that lack an affinity for water; tending to repel and not absorb water as well as not dissolve in or mix with water.

As used herein, an “oil” is a liquid or liquefiable material that is immiscible in water. In one embodiment, an oil is a composition containing at least 95% by weight of a lipophilic substance. Exemplary lipophilic substances include, but are not limited to, naturally occurring and synthetic oils, fats, fatty acids, lecithins, triglycerides and combinations thereof.

As used herein, an “emulsion” is a composition containing a mixture of non-miscible components homogenously blended together. In particular embodiments, the non-miscible components include a lipophilic component and an aqueous component. An emulsion is a preparation of one liquid (e.g., the discrete or discontinuous phase) distributed in small globules throughout the body of a second liquid (e.g., the continuous phase). When oil is the dispersed liquid and an aqueous solution is the continuous phase, it is known as an oil-in-water emulsion. When water or an aqueous solution is the dispersed phase and oil or oleaginous substance is the continuous phase, it is known as a water-in-oil emulsion. Either or both of the oil phase and the aqueous phase may contain one or more surfactants, emulsifiers, emulsion stabilizers, buffers, and other excipients. Preferred excipients include surfactants, especially non-ionic surfactants; emulsifying agents, especially emulsifying waxes; and liquid non-volatile non-aqueous materials, particularly glycols such as propylene glycol. The oil phase may contain other oily pharmaceutically approved excipients. For example, materials such as hydroxylated castor oil or sesame oil may be used in the oil phase as surfactants or emulsifiers.

As used herein, “emollients” are an externally applied agent that softens or soothes skin. Emollients are generally known in the art and listed in compendia, such as the “Handbook of Pharmaceutical Excipients”, 4^{th} Ed., Pharmaceutical Press, 2003. These include, without limitation, almond oil, castor oil, ceratonia extract, cetostearoyl alcohol, cetyl alcohol, cetyl esters wax, cholesterol, cottonseed oil, cyclomethicone, ethylene glycol palmitostearate, glycerin, glycerin monostearate, glyceryl monooleate, isopropyl myristate, isopropyl palmitate, lanolin, lecithin, light mineral oil, medium-chain triglycerides, mineral oil and lanolin alcohols, petrolatum, petrolatum and lanolin alcohols, soybean oil, starch, stearyl alcohol, sunflower oil, xylitol and combinations thereof. In one embodiment, the emollients are ethylhexylstearate and ethylhexyl palmitate.

As used herein, “surfactants” are surface-active agents that lower surface tension and thereby increase the emulsifying, foaming, dispersing, spreading and wetting properties of a product. Suitable non-ionic surfactants include emulsifying wax, glyceryl monooleate, polyoxyethylene alkyl ethers, polyoxyethylene castor oil derivatives, polysorbate, sorbitan esters, benzyl alcohol, benzyl benzoate, cyclodextrins, glycerin monostearate, poloxamer, povidone and combinations thereof. In one embodiment, the non-ionic surfactant is stearyl alcohol.

As used herein, “emulsifiers” are surface active substances which promote the suspension of one liquid in another and promote the formation of a stable mixture, or emulsion, of oil and water. Common emulsifiers are: metallic soaps, certain animal and vegetable oils, and various polar compounds. Suitable emulsifiers include acacia, anionic emulsifying wax, calcium stearate, carbomers, cetostearyl alcohol, cetyl alcohol, cholesterol, diethanolamine, ethylene glycol palmitostearate, glycerin monostearate, glyceryl monooleate, hydroxpropyl cellulose, hypromellose, lanolin, hydrous, lanolin alcohols, lecithin, medium-chain triglycerides, methylcellulose, mineral oil and lanolin alcohols, monobasic sodium phosphate, monoethanolamine, nonionic emulsifying wax, oleic acid, poloxamer, poloxamers, polyoxyethylene alkyl ethers, polyoxyethylene castor oil derivatives, polyoxyethylene sorbitan fatty acid esters, polyoxyethylene stearates, propylene glycol alginate, self-emulsifying glyceryl monostearate, sodium citrate dehydrate, sodium lauryl sulfate, sorbitan esters, stearic acid, sunflower oil, tragacanth, triethanolamine, xanthan gum and combinations thereof. In one embodiment, the emulsifier is glycerol stearate.

As used herein, a “lotion” is a suspension of an insoluble powder in a liquid or an emulsion having a viscosity of between 100 and 1000 centistokes.

As used herein, a “cream” is an emulsion having a viscosity of greater than 1000 centistokes, typically in the range of 20,000-50,000 centistokes.

As used herein, a “paste” is a liquid or emulsion having solid material homogenously suspended therein, typically in a lotion cream or gel.

As used herein, a “gel” is a composition containing a thickening agent or polymeric material dissolved or suspended in a liquid. The liquid may include a lipophilic component, an aqueous component or both. Some emulsions may be gels or otherwise include a gel component. Some gels, however, are not emulsions because some do not contain a homogenized blend of immiscible components.

“Penetration enhancers”, are used herein, refers to materials or techniques which promote transdermal delivery of drugs across the skin, in particular across the stratum corneum. These can be chemical penetration enhancers or physical penetration enhancers, such as ultrasound.

As used herein, the term “prodrug” refers to an active drug chemically transformed into an inactive derivative which, by virtue of chemical or enzymatic attack, is converted to the parent drug within the body before or after reaching the site of action. Prodrugs are frequently (though not necessarily) pharmacologically inactive until converted to the parent drug. A prodrug may be converted into the parent drug by various mechanisms, including enzymatic processes and metabolic hydrolysis. Examples of prodrugs include, but are not limited to, ester and amide prodrugs; polyethylene glycol prodrugs (with and without a linker); N-acyl amine prodrugs, dihydropyridine prodrugs, 2-hydroxybenzamide prodrgus; carbamate prodrugs; peptide prodrugs; Mannich bases, and Schiff bases.

As used herein, the term “analogue” refers to a chemical compound with a structure similar to that of another (reference compound) but differing from it in respect to a particular component, functional group, atom, etc.

As used herein, the term “derivative” refers to compounds which are formed from a parent compound by chemical reaction(s).

### II. Formulations

The formulations are designed for local treatment of the glans penis to produce local anesthesia of the dorsal nerves of the glans penis. The formulations can consist of one or more local anesthetics combined with one or more pharmaceutically acceptable excipients. The formulation can also further include other constituents such as penetration enhancers or other active agents. The formulations can contain active agents in the form of micro or nanoparticles, which may be formed of active agent alone or in combination with an excipient or carrier. The drug formulation may be administered as an emulsion, lotion, paste, gel, cream, ointment, shea butter, suspension, solution, balm, salve, or foam. The active agent formulation may also be administered as a pump spray.

A. Local Anesthetics

The compositions described herein contain one or more local anesthetics. Suitable local anesthetics include, but are not limited to, prilocalne, lidocaine, bupivacaine, chlorprocaine, dibucaine, etidocaine, mepivacaine, tetracaine, dyclonine, hexylcaine, procaine, ketamine, pramoxine, phenol, and combinations thereof.

In one embodiment, the composition contains lidocaine, prilocalne, or a mixture thereof. Prilocalne is a local anesthetic drug which has the chemical formula:

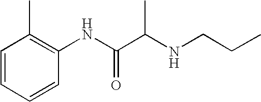

Prilocalne is described in British Patent 839,943 (1960 to Astra), and takes the form of crystalline needles having a melting point of 37° C.-38° C.

The hydrochloride salt, having the formula C₃H₂₁ClN₂O, is crystallized from ethanol and isopropyl ether, and is readily soluble in water.

Lidocaine is a local anesthetic drug which has the chemical formula:

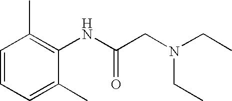

Lidocaine has the chemical formula C₁₄H₂₂N₂O.

The local anesthetic can be administered as the free acid or free base or as a pharmaceutically acceptable salt. As used herein, “pharmaceutically acceptable salts” refer to derivatives of the disclosed compounds wherein the parent compound is modified by making acid-addition or base-addition salts thereof. Examples of pharmaceutically acceptable salts include, but are not limited to, mineral or organic acid salts of basic residues such as amines; alkali or organic salts of acidic residues such as carboxylic acids. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, and isethionic.

The pharmaceutically acceptable salts of the compounds can be synthesized from the parent compound, which contains a basic or acidic moiety, by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, ethanol, isopropanol, or acetonitrile are preferred. Lists of suitable salts are found in Remington's Pharmaceutical Sciences, 20th ed., Lippincott Williams & Wilkins, Baltimore, Md., 2000, p. 704.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

In one embodiment, the local anesthetic is a mixture of lidocaine and prilocalne. Separately, lidocaine and prilocalne are solid bases. When mixed together in equal quanitites by weight, however, they form a eutectic mixture. A “eutetic mixture” is defined herein as a mixture in which the melting point of the mixture is lower than the melting points of the individual components. The lidocaine/prilocalne eutectic mixture is an oil with a melting point of 16° C., which is lipid soluble. This property allows this anesthetic drug mixture to be formulated into preparations without the use of a non-aqueous solvent. This allows higher concentrations of anaesthetic to be formulated into the preparation and maintained during application. Further, liquid forms of the drug are desirable in clinical applications as they are less likely to clog the metered dose valve of the administration device, unlike dry powders.

Lidocaine and prilocalne can be mixed together in any ratio. In one embodiment, lidocaine and prilocalne are mixed together in equal (50:50) ratios. In other embodiments, lidocaine and prilocalne are mixed together in 5:95, 10:90, 15:85 20:80, 25:75, 30:70, 35:65, 40:60, 45:65, 55:45, 60:40, 65:35, 70:30, 75:25, 80:20, 85:15, 90:10 and 95:5 ratios.

The concentration of the anesthetic drug in the formulation can vary up to 90% by weight, about 70% by weight, about 50% by weight, about 30% by weight, about 20% by weight, about 10% by weight, about 8% by weight, about 6% by weight, about 5% by weight, about 4% by weight, about 2% by weight, or about 1% by weight, of the formulation.

B. Other Active Agents

The compositions optionally contain one or more additional pharmaceutically active agents. Suitable classes of active agents include, but are not limited to, antimicrobial agents (antibacterial and antifungal agents, and/or antiprotozoal agents), anti-inflammatory agents (non-steroidal or steroidal agents), vasodilator agents, anti-oxidants, vitamins, and hormones.

Additional agents include benzoyl peroxide, zinc, azelaic acid and its derivatives, phenoxy ethanol and phenoxy proponol, ethylacetate, sebostats such as flavinoids; alpha and beta hydroxy acids; and bile salts such as scymnol sulfate and its derivatives, deoxycholate and cholate. The antibiotic can be an antifungal agent. Suitable antifungal agents include, but are not limited to, clotrimazole, econazole, ketoconazole, itraconazole, miconazole, oxiconazole, sulconazole, butenafine, naftifine, terbinafine, undecylinic acid, tolnaftate, and nystatin.

Suitable antimicrobial agents include, but are not limited to, antibacterial, antifungal, antiprotozoal and antiviral agents, such as beta-lactam drugs, quinolone drugs, ciprofloxacin, norfloxacin, a tetracycline, erythromycin, amikacin, triclosan, doxycycline, capreomycin, chlorhexidine, such as chlortetracycline, methacycline, oxytetracycline, clindamycin, ethambutol, metronidazole, pentamidine, gentamicin, kanamycin, lineomycin, methacycline, methenamine, minocycline, neomycin, netilmicin, streptomycin, tobramycin, and miconazole. Also included are tetracycline hydrochloride, famesol, erythromycin estolate, erythromycin stearate (salt), amikacin sulfate, doxycycline hydrochloride, chlorhexidine gluconate, chlorhexidine hydrochloride, chlortetracycline hydrochloride, oxytetracycline hydrochloride, clindamycin hydrochloride, ethambutol hydrochloride, metronidazole hydrochloride, pentamidine hydrochloride, gentamicin sulfate, kanamycin sulfate, lineomycin hydrochloride, methacycline hydrochloride, methenamine hippurate, methenamine mandelate, minocycline hydrochloride, neomycin sulfate, netilmicin sulfate, paromomycin sulfate, streptomycin sulfate, tobramycin sulfate, miconazole hydrochloride, amanfadine hydrochloride, amanfadine sulfate, triclosan, octopirox, nystatin, tolnaftate, clotrimazole, anidulafungin, micafungin, voriconazole, lanoconazole, ciclopirox, econazole, and mixtures thereof.

Representative examples of non-steroidal anti-inflammatory agents include, without limitation, oxicams, such as piroxicam, isoxicam, tenoxicam, sudoxicam; salicylates, such as aspirin, disalcid, benorylate, trilisate, safapryn, solprin, diflunisal, and fendosal; acetic acid derivatives, such as diclofenac, fenclofenac, indomethacin, sulindac, tolmetin, isoxepac, furofenac, tiopinac, zidometacin, acematacin, fentiazac, zomepirac, clindanac, oxepinac, felbinac, and ketorolac; fenamates, such as mefenamic, meclofenamic, flufenamic, niflumic, and tolfenamic acids; propionic acid derivatives, such as ibuprofen, naproxen, benoxaprofen, flurbiprofen, ketoprofen, fenoprofen, fenbufen, indopropfen, pirprofen, carprofen, oxaprozin, pranoprofen, miroprofen, tioxaprofen, suprofen, alminoprofen, and tiaprofenic; pyrazoles, such as phenylbutazone, oxyphenbutazone, feprazone, azapropazone, and trimethazone. Mixtures of these non-steroidal anti-inflammatory agents may also be employed, as well as the dennatologically acceptable salts and esters of these agents. For example, etofenamate, a flufenamic acid derivative, is particularly useful for local application.

Representative examples of steroidal anti-inflammatory drugs include, without limitation, corticosteroids such as hydrocortisone and hydrocortisone butyrate, hydroxyl-triamcinolone, alpha-methyl dexamethasone, amcinafel, amcinafide, beclomethasone dipropionates, betamethasone and the balance of its esters, chloroprednisone, chlorprednisone acetate, clescinolone, clobetasol valerate, clocortelone, cortisone, cortodoxone, dexamethasone-phosphate, desonide, desoxymethasone, desoxycorticosterone acetate, dexamethasone, dichlorisone, diflorasone diacetate, diflucortolone valerate, fluclorolone acetonide, fludrocortisone, flumethasone pivalate, fluosinolone acetonide, fluocinonide, flucortine butylesters, fluocortolone, fluprednidene (fluprednylidene) acetate, flurandrenolone, halcinonide, hydrocortisone acetate, methylprednisolone, triamcinolone acetonide, flucetonide, fludrocortisone, fluradrenolone, fludrocortisone, difluorosone diacetate, fluradrenolone acetonide, medrysone, diflurprednate, flucloronide, flunisolide, fluoromethalone, fluperolone, fluprednisolone, hydrocortisone valerate, hydrocortisone cyclopentylpropionate, hydrocortamate, meprednisone, paramethasone, prednisolone, prednisone, beclomethasone dipropionate, triamcinolone, and mixtures thereof.

One or more vasodilators may also be added to the formulations. Vasodilators are known to produce and maintain penile erection whether given alone or in combination with other vasodilators. Typically, vasodilator agents are injected into the erectile tissue, however this method of administration is painful and puts the patient at risk of nerve damage and/or excessive bleeding. See, for example, U.S. Pat. Nos. 6,007,836 and 5,256,652. Suitable vasodilator agents include, but are not limited to alpha-adrenoceptor antagonists (alpha-blockers), angiotensin converting enzyme inhibitors (ACE inhibitors), angiotensin receptor blockers (ABs), beta₂-adrenoceptor agonists (β₂-agonists), calcium-channel blockers, endothelin receptor antagonists, nitrodilators, phosphodiesterase (PDE) inhibitors and potassium-channel openers.

Representative examples of alpha-blockers include, without limitation, prazosin, terazosin, doxazosin, trimazosin, phentolamine and phenoxybenzamine.

Representative examples of ACE inhibitors include, without limitation, benazepril, captopril, enalapril, fosinopril, lisinopril, moexipril, quinapril and ramipril.

Representative examples of ARBs include, without limitation, candesartan, eprosartan, irbesartan, losartan, olmesartan, telmisartan and valsartan.

Representative examples of β₂-agonists include, without limitation, epinephrine, norepinephrine, dopamine, doputamine and isoproterenol.

Representative examples of calcium-channel inhibitors include, without limitation, dihydropyridines such as amlodipine, felodipine, isradipine, nicardipine, nifedipine, nimodipine and nitrendipine, and non-dihydropyridines, such as verapamil and diltiazem.

Representative examples of endothelin receptor antagonists include, without limitation, ambrisentan, sitaxsentan and bosentan.

Representative examples of nitrodilators include, without limitation, nitric oxide, isosorbide dinitrate, isosorbide mononitrate, nitroglycerin, erythrityl tetranitrate, pentaerythritol tetranitrate and sodium nitroprusside.

Representative examples of phosphodiesterase inhibitors include, without limitation, milrinone, amrinone, sildenafil and tadalafil.

Representative examples of potassium channel openers include, without limitation, mioxidil, levosimendan and levcromakalim. Additional vasodilator agents, include, but are not limited to, papaverine, prostaglandin E-1, dioyline and ethaverine.

C Carriers, Excipients, Solvents and Additives

The one or more local anesthetics are administered in a carrier or a pharmaceutically acceptable carrier for local administration. Suitable carriers or excipients include, but are not limited to, emulsifiers, diluents, surfactants, solubility enhancers, suspending agents, anti-oxidants, chelating agents, emollients, humectants, pH modifying agents, lipid bilayer disrupting agents, preservatives, thickening agents, viscosity modifying agents, vitamins and other skin nutrients, and combinations thereof. Other additives and excipients include, without limitation, viscosifiers, additional occluding agents, fragrances, deodorants, colorants, preservatives, vitamins and other skin nutrients, antioxidants, solubility enhancers, and other stabilizing agents. The various components described above can be collected and provided as a kit. Additionally, the one or more local anesthetics may be dissolved in saline solution (e.g., 0.9% saline or physiological saline solution), water or an alcohol solution.

i. Diluents

Diluents may be included in the formulations to dissolve, disperse or otherwise incorporate the drug into the carrier. Examples of diluents include, but are not limited to, water, buffered aqueous solutions, organic hydrophilic diluents, such as monovalent alcohols, and low molecular weight glycols and polyols (e.g. propylene glycol, polypropylene glycol, glycerol, butylene glycol).

ii. Emollients

Suitable emollients include petrolatum, high-melting fatty acids and esters, high-melting triglycerides, lanolin, hydrogenated castor oil, hydroxyethylated castor oil, and hydrogenated hydroxyethylated castor oil, in each case when their melting point is above about 38° C. Additional emollients are well known, and listings can be found can be found in reference books, for example under “Skin Conditioning Agents—Emollient” and “Skin Conditioning Agents—Occlusive” in the “CFTA Cosmetic Ingredient Handbook”, copyright 1988 by the Cosmetics, Toiletries and Fragrance Association of Washington, D.C. Any of the known approved emollients is potentially suitable for use in the composition if it melts above body temperature. Mixtures of emollients can be used, Concentration ranges of between 1 and 15 weight percent are possible with a range of between 2.5 to 11 weight percent preferred. Other USP-grade oils can be used if they are liquid at or near body temperature, preferably at room temperature. These materials may be conventional vegetable oils, such as corn, canola, peanut, soy, olive, or other plant extracts. Other types of oil include silicone oils and mineral (hydrocarbon) oils. Combinations of oils with each other can also be used.

iii. Emulsifiers

An emulsifier is desirable to help promote efficient release of the actives from the formulation. Suitable surfactants include, but are not limited to, anionic surfactants, non-ionic surfactants, cationic surfactants, and amphoteric surfactants. Other suitable emulsifiers include, but are not limited to, straight chain or branched fatty acids, polyoxyethylene sorbitan fatty acid esters, sorbitan fatty acid esters, propylene glycol stearate, glyceryl stearate, polyethylene glycol, fatty alcohols, polymeric ethylene oxide-propylene oxide block copolymers, and combinations thereof.

iv. Surfactants

Examples of anionic surfactants include, but are not limited to, ammonium lauryl sulfate, sodium lauryl sulfate, ammonium laureth sulfate, sodium laureth sulfate, alkyl glyceryl ether sulfonate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, potassium lauryl sulfate, potassium laureth sulfate, sodium lauryl sarcosinate, sodium lauroyl sarcosinate, lauryl sarcosine, cocoyl sarcosine, ammonium cocoyl sulfate, ammonium lauroyl sulfate, sodium cocoyl sulfate, sodium lauroyl sulfate, potassium cocoyl sulfate, potassium lauryl sulfate, triethanolamine lauryl sulfate, triethanolamine lauryl sulfate, monoethanolamine cocoyl sulfate, monoethanolamine lauryl sulfate, sodium tridecyl benzene sulfonate, sodium dodecyl benzene sulfonate, sodium and ammonium salts of coconut alkyl triethylene glycol ether sulfate; tallow alkyl triethylene glycol ether sulfate, tallow alkyl hexaoxyethylene sulfate, disodium N-octadecylsulfosuccinnate, disodium lauryl sulfosuccinate, diammonium lauryl sulfosuccinate, tetrasodium N-(1,2-dicarhoxyethyl)-N-octadecylsulf-osuccinnate, diamyl ester of sodium sulfosuccinic acid, dihexyl ester of sodium sulfosuccinic acid, dioctyl esters of sodium sulfosuccinic acid, docusate sodium, and combinations thereof.

Examples of nonionic surfactants include, but are not limited to, polyoxyethylene fatty acid esters, sorbitan esters, cetyl octanoate, cocamide DEA, cocamide MEA, cocamido propyl dimethyl amine oxide, coconut fatty acid diethanol amide, coconut fatty acid monoethanol amide, diglyceryl diisostearate, diglyceryl monoisostearate, diglyceryl monolaurate, diglyceryl monooleate, ethylene glycol distearate, ethylene glycol monostearate, ethoxylated castor oil, glyceryl monoisostearate, glyceryl monolaurate, glyceryl monomyristate, glyceryl monooleate, glyceryl monostearate, glyceryl tricaprylate/caprate, glyceryl triisostearate, glyceryl trioleate, glycol distearate, glycol monostearate, isooctyl stearate, lauramide DEA, lauric acid diethanol amide, lauric acid monoethanol amide, lauric/myristic acid diethanol amide, lauryl dimethyl amine oxide, lauryl/myristyl amide DEA, lauryl/myristyl dimethyl amine oxide, methyl gluceth, methyl glucose sesquistearate, oleamide DEA, PEG-distearate, polyoxyethylene butyl ether, polyoxyethylene cetyl ether, polyoxyethylene lauryl amine, polyoxyethylene lauryl ester, polyoxyethylene lauryl ether, polyoxyethylene nonylphenyl ether, polyoxyethylene octyl ether, polyoxyethylene octylphenyl ether, polyoxyethylene oleyl amine, polyoxyethyelen oleyl cetyl ether, polyoxyethylene oleyl ester, polyoxyethylene oleyl ether, polyoxyethylene stearyl amine, polyoxyethylene stearyl ester, polyoxyethylene stearyl ether, polyoxyethylene tallow amine, polyoxyethylene tridecyl ether, propylene glycol monostearate, sorbitan monolaurate, sorbitan monooleate, sorbitan monopalmitate, sorbitan monostearate, sorbitan sesquioleate, sorbitan trioleate, stearamide DEA, stearic acid diethanol amide, stearic acid monoethanol amide, laureth-4, Oleth-10 (polyoxyethylene (10) oleyl ether) and combinations thereof.

Examples of amphoteric surfactants include, but are not limited to, sodium N-dodecyl-ÿ-alanine, sodium N-lauryl-ÿ-iminodipropionate, myristoamphoacetate, lauryl betaine, lauryl sulfobetaine, sodium 3-dodecyl-aminopropionate, sodium 3-dodecylaminopropane sulfonate, sodium lauroamphoacetate, cocodimethyl carboxymethyl betaine, cocoamidopropyl betaine, cocobetaine, lauryl amidopropyl betaine, oleyl betaine, lauryl dimethyl carboxymethyl betaine, lauryl dimethyl alphacarboxyethyl betaine, cetyl dimethyl carboxymethyl betaine, lauryl bis-(2-hydroxyethyl)carboxymethyl betaine, stearyl bis-(2-hydroxypropyl)carboxymethyl betaine, oleyl dimethyl gamma-carboxypropyl betaine, lauryl bis-(2-hydroxypropyl)alpha-carboxyethyl betaine, oleamidopropyl betaine, coco dimethyl sulfopropyl betaine, stearyl dimethyl sulfopropyl betaine, lauryl dimethyl sulfoethyl betaine, lauryl bis-(2-hydroxyethyl)sulfopropyl betaine, and combinations thereof.

Examples of cationic surfactants include, but are not limited to, behenyl trimethyl ammonium chloride, bis(acyloxyethyl)hydroxyethyl methyl ammonium methosulfate, cetrimonium bromide, cetrimonium chloride, cetyl trimethyl ammonium chloride, cocamido propylamine oxide, distearyl dimethyl ammonium chloride, ditallowdimonium chloride, guar hydroxypropyltrimonium chloride, lauralkonium chloride, lauryl dimethylamine oxide, lauryl dimethylbenzyl ammonium chloride, lauryl polyoxyethylene dimethylamine oxide, lauryl trimethyl ammonium chloride, lautrimonium chloride, methyl-1-oleyl amide ethyl-2-oleyl imidazolinium methyl sulfate, picolin benzyl ammonium chloride, polyquaternium, stearalkonium chloride, sterayl dimethylbenzyl ammonium chloride, stearyl trimethyl ammonium chloride, trimethylglycine, and combinations thereof. Those skilled in the art would be able to test other surfactants, beginning with those having similar HLB, in order to arrive to stable formulations. Mixtures of surfactants can be used to optimize the properties of the formulation.

v. Solubility Enhancers

Suitable solubility enhancing agents include solvents such as water; diols, such as propylene glycol and glycerol; mono-alcohols, such as ethanol, propanol, and higher alcohols; DMSO; dimethylformamide; N,N-dimethylacetamide; 2-pyrrolidone; N-(2-hydroxyethyl)pyrrolidone, N-methylpyrrolidone, 1-dodecylazacycloheptan-2-one and other n-substituted-alkyl-azacycloalkyl-2-ones and other n-substituted-alkyl-azacycloalkyl-2-ones (azones).

vi. Penetration Enhancers

Penetration enhancers are frequently used to promote transdermal delivery of drugs across the skin, in particular across the stratum corneum. Some penetration enhancers cause dermal irritation, dermal toxicity and dermal allergies. Suitable penetration enhancers include, but are not limited to, urea, (carbonyldiamide), imidurea, A, N-diethylformamide, N-methyl-2-pyrrolidine, 1-dodecal-azacyclopheptane-2-one, calcium thioglycate, 2-pyyrolidine, N,N-diethyl-m-toluamide, oleic acid and its ester derivatives, such as methyl, ethyl, propyl, isopropyl, butyl, vinyl and glycerylmonooleate, sorbitan esters, such as sorbitan monolaurate and sorbitan monooleate, other fatty acid esters such as isopropyl laurate, isopropyl myristate, isopropyl palmitate, diisopropyl adipate, propylene glycol monolaurate, propylene glycol monooleate and non-ionic detergents such as BRIJ® 76 (stearyl poly(10 oxyethylene ether), BRIJ® 78 (stearyl poly(20)oxyethylene ether), BRIJ® 96 (oleyl poly(10)oxyethylene ether), and BRIJ® 721 (stearyl poly (21) oxyethylene ether) (ICI Americas Inc. Corp.). Fatty acids such as linoleic acid, capric acid, lauric acid, and neodecanoic acid, which can be in a solvent such as ethanol or propylene glycol, can be used as lipid bilayer disrupting agents.

vii. Suspending Agents

Suitable suspending agents include, but are not limited to, alginic acid, bentonite, carbomer, carboxymethylcellulose and salts thereof, hydroxyethylcellulose, hydroxypropylcellulose, microcrystalline cellulose, colloidal silicon dioxide, dextrin, gelatin, guar gum, xanthan gum, kaolin, magnesium aluminum silicate, maltitol, triglycerides, methylcellulose, polyoxyethylene fatty acid esters, polyvinylpyrrolidone, propylene glycol alginate, sodium alginate, sorbitan fatty acid esters, tragacanth, and combinations thereof.

viii. Antioxidants

Suitable antioxidants include, but are not limited to, butylated hydroxytoluene, alpha tocopherol, ascorbic acid, fumaric acid, malic acid, butylated hydroxyanisole, propyl gallate, sodium ascorbate, sodium metabisulfite, ascorbyl palmitate, ascorbyl acetate, ascorbyl phosphate, Vitamin A, folic acid, flavons or flavonoids, histidine, glycine, tyrosine, tryptophan, carotenoids, carotenes, alpha-Carotene, beta-Carotene, uric acid, pharmaceutically acceptable salts thereof, derivatives thereof, and combinations thereof.

ix. Chelating Agents

Suitable chelating agents include, but are not limited to, EDTA, disodium edetate, trans-1,2-diaminocyclohexane-N,N>N′,N′-tetraacetic acid monohydrate, N,N-bis(2-hydroxyethyl)glycine, 1,3-diamino-2-hydroxypropane-N,N,N′,N′-tetraacetic acid, 1,3-diaminopropane-N,N,N′,N′-tetraacetic acid, ethylenediamine-N,N′-diacetic acid, ethylenediainine-N,N′-dipropionic acid, ethylenediamine-N,N′-bis(methylenephosphonic acid), N-(2-hydroxyethyl)ethylenediamine-N,N′,N′-triacetic acid, ethylenediamine-N,N,N′,N′-tetrakis(methylenephosphonic acid), O,O′-bis(2-aminoethyl)ethyleneglycol-N,N,N′,N′-tetraacetic acid, N,N-bis(2-hydroxybenzyl)ethylenediamine-N,N-diacetic acid, 1,6-hexamethylenediamine-N,N,N′,N′-tetraacetic acid, N-(2-hydroxyethyl)iminodiacetic acid, iminodiacetic acid, 1,2-diaminopropane-N,N,N′,N′-tetraacetic acid, nitrilotriacetic acid, nitrilotripropionic acid, nitrilotris(methylenephosphoric acid), 7,19,30-trioxa-1,4,10,13,16,22,27,33-octaazabicyclo[11,11,11]pentatriacontane hexahydrobromide, triethylenetetramine-N,N,N′,N″,N′″,N′″-hexaacetic acid, and combinations thereof.

x. Humectants

Suitable humectants include, but are not limited to, glycerin, butylene glycol, propylene glycol, sorbitol, triacetin, and combinations thereof.

xi. pH Modifying Agents

The compositions described herein may further contain a pH modifying agent including, but are not limited to, sodium hydroxide, citric acid, hydrochloric acid, acetic acid, phosphoric acid, succinic acid, sodium hydroxide, potassium hydroxide, ammonium hydroxide, magnesium oxide, calcium carbonate, magnesium carbonate, magnesium aluminum silicates, malic acid, potassium citrate, sodium citrate, sodium phosphate, lactic acid, gluconic acid, tartaric acid, 1,2,3,4-butane tetracarboxylic acid, fumaric acid, diethanolamine, monoethanolamine, sodium carbonate, sodium bicarbonate, triethanolamine, and combinations thereof.

xii. Preservatives.

Preservatives can be used to prevent the growth of fungi and other microorganisms. Suitable preservatives include, but are not limited to, benzoic acid, butylparaben, ethyl paraben, methyl paraben, propylparaben, sodium benzoate, sodium propionate, benzalkonium chloride, benzethonium chloride, benzyl alcohol, cetypyridinium chloride, chlorobutanol, phenol, phenylethyl alcohol, thimerosal, and combinations thereof.

D. Dosage Forms

The local anesthetic can be formulated as an emulsion, lotion, paste, gel, cream, ointment, shea butter, balm, salve or foam using techniques well known in the art. Ansel et al, Pharmaceutical Dosage Forms and Drug Delivery Systems, 6^{th} Ed. Williams and Williams (1995) describes the preparation of a variety of dosage forms for topical and transdermal administration of one or more active agents.

1. Solutions, Dispersions, Suspensions, Emulsions, Butters

Methods for making topical carriers are well known, Many different carriers can be utilized. Suitable excipient bases can be purchased from formulators, to which an effective amount of active agent is added. These may be excipients for forming a solution, dispersion or suspension of drug or drug particles, emulsifiers and surfactants for making an emulsion, or lipids and surfactants for making shea butters and other similar products.

2. Gels, Pastes

An emulsifier is desirable to help promote efficient release of the active agents from the formulation. A wide variety of surfactants are potentially useful. Useful nonionic surfactants include Oleth-10 (polyoxyethylene (10) oleyl ether) in a range from about 1% to about 15%. Those skilled in the art would be able to test other surfactants, beginning with those having similar HLB, in order to arrive to stable formulations. Mixtures of surfactants can be used to optimize the properties of the formulation.

Any pharmaceutically or cosmetically acceptable thickener, for example, those suitable for thickening hydrocarbon, silicone or vegetable oils may be used in the formulations. The thickeners modify the rheology of the formulations in order to establish the proper balance between activity and, application and post application physical behavior. Examples of thickeners include colloidal silicas and starches. An example of a preferred thickener is colloidal silica. The thickener is used in a concentration range of between 1.0% to about 5.0%, more preferably in a range of between 1.0% and 2.5%. Those skilled in the art would be able to test other thickeners in order to prepare stable formulations. Mixtures of thickeners can be used to optimize the properties of the formulation. The gel or pastes may further contain a penetration enhancer.

4. Pump spray

The formulation can also be in the form of a propellant-free aerosol preparation such as a pump spray. In one embodiment, the formulation contains active agents dissolved in 0.9% saline solution, or alternatively, a physiologic saline solution. See Hoar and Hickman, A Companion for General and Comparative Physiology, 1975. In another embodiment, the formulation contains active agents dissolved in water.

In another embodiment, the formulation contains a liquid phase composed of aliphatic C₂-C₄ alcohols and a vehicle. In a preferred embodiment, the C₂-C₄ alcohol is ethyl alcohol and the carrier is a neutral oil. In one embodiment, the liquid phase includes 10 to 40% by weight of ethyl alcohol and 90 to 60% by weight of an oil. A pump spray of this composition produces a uniform mist and a sufficient quantity of the preparation or active substance is still available after a relatively long storage time. Even if, after 7 days storage, a quantity loss of maximum 10%, based on the active substance solution present in the metering chamber, is recorded in the first squirt, the loss of active substance is smaller because the latter remains dissolved in the less readily evaporable neutral oil.

The active agent or agents may be provided in an approximately 5% oily solution and then mixed with the other requisite quantities of neutral oil and optionally with other additives and transferred to the pump spray bottles with the addition of ethyl alcohol.

In one embodiment, the fatty oils are neutral oils such as synthetic triglycerides whose fatty acid proportion is composed of saturated C₈ to C₁₂ fatty acids. These triglycerides are also known as Miglyol types. Various types of Miglyol are distinguished according to the caprylic acid (C₈:0) and capric acid (C₁₀:0) content. In another embodiment, natural oils are used. Preferred natural oils include those oils that contain as few unsaturated fatty acids as possible. This can be achieved by hydrogenation in the case of oils which contain a large quantity of unsaturated fatty acids. The proportion of the neutral oil in the pump spray can be 90 to 60% by weight, but is usually 85 to 70% by weight. Neutral oil quantities of about 80% by weight are preferred. In another embodiment, the formulation contains a mixture of lidocaine and prilocalne dissolved in an aqueous saline solution. The pump spray can contain other additives.

The proportion of active agent in the formulation can vary but is usually between 0.1 and 32% by weight. For example, approximately a 1:3 ratio of prilocalne to lidocaine is suitable for maintaining both anesthetics in liquid form and providing the desired desensitization without affecting the surrounding tissue or the partner. In one embodiment, the amount of prilocalne in the formulation can range from about 2 percent to about 8 percent by weight. In another embodiment, the amount of prilocalne may range from about 3 percent to about 6% by weight, or from about 4 percent to about 5% by weight. The amount of lidocaine may range from about 6 percent to about 24 percent by weight, or from about 8 percent to about 18 percent, or from about 10 percent to about 14 percent, by weight.

In one embodiment, the formulation includes about 7.5 mg of prilocalne and about 22.5 mg of lidocaine applied prior to intercourse. This dose may be divided for easier and more effective application. For example, a pump spray applicator may be tuned or metered to administer about 2.5 milligrams of prilocalne and about 7.5 mg of lidocaine per spray. This would allow the user to cover the entire glans in three sprays.

### III. Methods of Administration

The formulations are administered locally, preferably onto the glans penis. The local formulation may be left on the glans penis or may be removed after an anesthetic effect is achieved. The formulation may be administered prior to or at the time of intercourse, but preferably prior to and any remainder removed or covered by a prophylactic.

The combination anesthetic formulation may be administered at a total dose of between about 20 mg to about 40 mg, preferably between about 25 mg to about 35 mg, more preferably from about 25 to about 30 mg, of anesthetic. This dose is effective to induce desensitization of the dorsal nerves of the penis. Several actuations of a pump spray device containing active agents, including local anesthetics, dissolved in saline, water, or an alcohol is a suitable method of delivery.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

We claim:

1. A formulation for treating premature ejaculation comprising a pharmaceutically acceptable carrier for topical administration to the glans comprising at least one local anesthetic in an amount effective to achieve desensitization of the dorsal nerves of the penis.

2. The formulation of claim 1 in a dosage form selected from the group consisting of an emulsion, lotion, paste, gel, cream, ointment, shea butter, suspension, solution, balm, salve, foam, or pump spray.

3. The formulation of claim 1 wherein the at least one local anesthetic is selected from the group consisting of lidocaine, prilocalne, bupivacaine, chlorprocaine, dibucaine, etidocaine, mepivacaine, tetracaine, dyclonine, hexylcaine, procaine, ketamine, pramoxine and phenol, a derivative or prodrug thereof, and combinations thereof.

4. The formulation of claim 1 wherein the at least one local anesthetic is a combination of prilocalne and lidocaine, or derivatives or prodrugs thereof.

5. The formulation of claim 4 wherein the pharmaceutically acceptable carrier comprises saline solution or water.

6. The formulation of claim 4 wherein the pharmaceutically acceptable carrier comprises an alcohol solution.

7. The formulation of claim 4 wherein the are present in about a 1:3 ratio of prilocalne to lidocaine.

8. The formulation of claim 1 in the form of a pump spray comprising from about 8 percent to about 32 percent by weight of the at least one anesthetic active agent, a liquid phase comprising from about 10 to about 40 percent by weight of ethyl alcohol and from about 60 to about 90 percent by weight of synthetic or natural fatty oils.

9. The formulation of claim 1 further comprising a vasodilator agent.

10. The formulation of claim 9 wherein the vasodilator agent comprises papaverine, phentolamine, prostaglandin E-1, dioxyline, ethaverine, or combinations thereof.

11. The formulation of claim 9 wherein the vasodilator agent is present in an amount of from about 1 percent to about 10 percent by weight of the composition.

12. A method for treating premature ejaculation comprising:
administering to a male human in need thereof a formulation comprising an effective amount of at least one local anesthetic, derivative or prodrug thereof in a pharmaceutically acceptable carrier for topical application as defined by any of claims 1-11 to the glans penis to achieve desensitization of the dorsal nerves of the penis.

13. The method of claim 12 wherein the at least one anesthetic is applied in amount ranging from 20 mg to 40 mg.

14. The method of claim 13 wherein the formulation is applied in an amount of about 30 mg.

15. The method of claim 14 wherein the at least one anesthetic comprises prilocalne and lidocaine in about a 1:3 ratio.

16. The method of claim 12 wherein the pharmaceutically acceptable carrier comprises saline solution or water.

17. The method of claim 12 wherein the pharmaceutically acceptable carrier comprises an alcohol solution.

18. The method of claim 12 wherein the step of administering the formulation comprises spraying the formulation using a pump spray applicator.

19. A method for treating premature ejaculation comprising:
topically administering to the glans penis of a male human in need thereof a formulation comprising from about 2 mg to about 8 mg of prilocalne and from about 6 mg to about 24 mg of lidocaine,
wherein the prilocalne and lidocaine are dissolved or suspended in a solvent selected from the group consisting of saline solution, water, alcohol, or combinations thereof and
wherein the step of topically administering the formulation comprises spraying the formulation using a metered pump spray applicator.

20. The method of claim 19 wherein the formulation further comprises a vasodilator agent selected from the group consisting of papaverine, phentolamine, prostaglandin E-1, dioxyline, ethaverine, or combinations thereof.
